(19) United States
(12) Patent Application Publication
(10) Pub. No.: US 2021/0228286 A1
(43) Pub. Date: Jul. 29, 2021

(54) SYSTEM AND METHOD FOR ASSISTING A USER IN A SURGICAL PROCEDURE

(71) Applicant: LIVE VUE TECHNOLOGIES INC., Kanata (CA)

(72) Inventors: Hassan Ghaderi MOGHADDAM, Kanata (CA); Mathieu DUPONT, Quebec (CA)

(21) Appl. No.: 17/053,851

(22) PCT Filed: May 10, 2019
(86) PCT No.: PCT/CA2019/050630

§ 371 Date: Nov. 9, 2020

Related U.S. Application Data
(60) Provisional application No. 62/669,496, filed on May 10, 2018.

(51) Int. Cl. A61B 34/00; G06T 19/00; A61B 34/10

(52) U.S. Cl. CPC A61B 34/25; G06T 19/006; A61B 2034/107; A61B 2034/254; A61B 34/10

(57) ABSTRACT

A system and a method are used for assisting a user in a surgical procedure. Three dimensional (3D) positioning information about an anatomical structure of interest and 3D positioning information about a surgical tool are acquired. An augmented reality view of spatial relations between the anatomical structure of interest and one or more positions along a path of the surgical tool is created based on the 3D positioning information. The augmented reality view is displayed superimposed over a field of view of the user. The augmented reality view may be displayed on a head-mounted display. Fiducial markers may be placed near the anatomical structure to provide enhanced positioning information to the creation of the augmented reality view. Operation of the surgical tool may be controlled in view of relative positions of the surgical tool and of a given landmark indicative of the anatomical structure.

### TECHNICAL FIELD

The present disclosure relates to the field of medical imaging. More specifically, the present disclosure relates to a system and a method for assisting a user in a surgical procedure.

### BACKGROUND

Medical imaging techniques using augmented reality are currently proposed for assisting surgical procedures, both in medical and dental fields. Three dimensional (3D) images are projected and superimposed on a field of view of a user, for example a clinician.

Conventional medical imaging techniques rely on 3D images of a patient obtained before a surgical procedure, for example by way of a computerized tomography (CT) scan. Clinicians use the augmented reality information as a guide to various anatomical features of the patient that may not be directly visible.

Conventional techniques are generally limited to presenting outer contours of some anatomical features, for example bones, which can be displayed overlaying the patient as seen by the clinician. As the clinician uses a surgical tool, for example a drill or a saw, to pierce or cut into an anatomical feature, for example a bone, little or no information is provided to the clinician about the position of the surgical tool or about the ongoing alterations of the anatomical feature resulting from the clinician's actions.

Therefore, there is a need for improvements to medical imaging techniques that compensate for problems related to the lack of details related to the position of surgical tools and to alterations of anatomical features in the course of surgical procedures.

### SUMMARY

The present disclosure introduces augmented reality techniques that may be used to assist a clinician, for example a dental surgeon or a medical surgeon, in the course of a surgical procedure. The clinician can follow in real time alterations of an anatomical structure of interest, i.e. the body part on which the surgical procedure is being applied, as well as the progression of a surgical tool used to perform the surgical procedure. In a particular aspect, the clinician may observe the position of a working end of a surgical tool, for example the tip of a drill bit, in relation to a landmark indicative of the anatomical structure of interest, for example a nerve or a blood vessel. Any portion of a medical image dataset and any visual element derived from a treatment plan dataset for the surgical procedure may be overlaid in a field of view of the clinician, allowing full and unhindered visualization of imaging information in an augmented reality environment.

In certain embodiments, this can allow the clinician to perform the surgery whilst avoiding certain anatomical structures such as nerves and blood vessels. This can minimize the chances of complications of the surgery and help towards maximizing the chances of success of the surgery. In certain embodiments, the overlaying of the treatment comprises overlaying an image of the final position of an implant based on a trajectory of the surgical tool in a tissue of the patient. The image of the implant final position can be updated in real-time such that the clinician can see in real-time the effect of the trajectory of the surgical tool. In this way, the clinician can make a decision regarding whether to continue with that trajectory or to abort or alter the trajectory.

According to the present disclosure, there is provided a system for assisting a user in a surgical procedure. The system comprises at least one sensor, a computer and a display. The at least one sensor is adapted to provide three dimensional (3D) positioning information about an anatomical structure of interest and about a surgical tool. The computer is operatively connected to the at least one sensor and adapted to determine a path of the surgical tool and create, based on the 3D positioning information, an augmented reality view of spatial relations between the anatomical structure of interest and one or more positions along a path of the surgical tool. The display device is adapted to display the augmented reality view superimposed over a field of view of the user.

In some implementations of the present technology, the computer is further adapted to determine the path of the surgical tool at least in part based on a previous position of the surgical tool.

In some implementations of the present technology, the computer is further adapted to determine the path of the surgical tool at least in part based on a predicted position of the surgical tool calculated based on a current position of the surgical tool and on a current orientation of the surgical tool.

In some implementations of the present technology, the at least one sensor is further adapted to provide 3D positioning information about a plurality of landmarks indicative of the anatomical structure of interest.

In some implementations of the present technology, the system further comprises at least one fiducial marker adapted for placement proximally to the anatomical structure of interest, the at least one sensor being further adapted to provide 3D positioning information about the at least one fiducial marker, and the computer being adapted to create the augmented reality view further based on the 3D positioning information about the at least one fiducial marker.

In some implementations of the present technology, the at least one fiducial marker is an infrared emitter and the at least one sensor comprises an infrared detector.

In some implementations of the present technology, the at least one fiducial marker comprises a plurality of fiducial markers, and the computer is further adapted to triangulate the 3D positioning information about the plurality of markers.

In some implementations of the present technology, one of the at least one fiducial marker is a 3D fiducial structure, and the at least one sensor is further adapted to provide 3D positioning information about a plurality of reference points on the 3D fiducial structure.

In some implementations of the present technology, the system further comprises a database operatively connected to the computer and storing a 3D map of the anatomical structure of interest and of the at least one fiducial marker, the computer being further adapted to create the augmented reality view based on the 3D map.

In some implementations of the present technology, the 3D map is obtained from an apparatus selected from one or more of a computerized tomography (CT) scanner, a cone beam computed tomography (CBCT) scanner and a magnetic resonance imaging (MRI) device.

In some implementations of the present technology, the at least one sensor comprises a 3D camera adapted to capture images of the anatomical structure of interest, of the at least one fiducial marker, and of the surgical tool, and the computer is further adapted to perform a registration between images captured by the 3D camera and a content of the 3D map to update the 3D map.

In some implementations of the present technology, the at least one sensor comprises a 3D camera adapted to capture images of the anatomical structure of interest, of the at least one fiducial marker, and of the surgical tool, and the computer is further adapted to generate the 3D map based on the captured images.

In some implementations of the present technology, the system further comprises a database operatively connected to the computer and storing a 3D map of the anatomical structure of interest, the computer being further adapted to create the augmented reality view based on the 3D map.

In some implementations of the present technology, the 3D map is obtained from an apparatus selected from one or more of a computerized tomography (CT) scanner, a cone beam computed tomography (CBCT) scanner and a magnetic resonance imaging (MRI) device.

In some implementations of the present technology, the at least one sensor comprises a 3D camera adapted to capture images of the anatomical structure of interest and of the surgical tool, and the computer is further adapted to perform a registration between images captured by the 3D camera and a content of the 3D map to update the 3D map.

In some implementations of the present technology, the at least one sensor comprises a 3D camera adapted to capture images of the anatomical structure of interest and of the surgical tool, and the computer is further adapted to generate the 3D map based on the captured images.

In some implementations of the present technology, the 3D map comprises a plurality of voxels distributed over three dimensions, each voxel having at least one intensity value and a coordinate over each of the three dimensions.

In some implementations of the present technology, each voxel has at least one polychromatic value, the at least one polychromatic value being derivable from the at least one intensity value.

In some implementations of the present technology, the 3D map comprises position, orientation and scale information of features of the anatomical structure of interest.

In some implementations of the present technology, the computer is further adapted to select elements of the 3D map representing at least one cross-section of the anatomical structure of interest, and create the augmented reality view further based on the at least one cross-section of the anatomical structure of interest.

In some implementations of the present technology, the system further comprises a control unit operatively connected to the computer and adapted to control an operation of the surgical tool.

In some implementations of the present technology, the at least one sensor is further adapted to provide, in real time, the 3D positioning information about the anatomical structure of interest and about the surgical tool, the computer is further adapted to provide, in real time, spatial relations between the one or more positions along the path of the surgical tool and a position of a given landmark indicative of the anatomical structure of interest to the control unit, and the control unit is further adapted to control, in real time, the operation of the surgical tool in view of the spatial relations between the one or more positions along the path of the surgical tool and the position of the given landmark indicative of the anatomical structure of interest.

In some implementations of the present technology, the surgical tool comprises a working end, the at least one sensor is further adapted to provide, in real time, positioning information about the working end to the computer, and the computer is further adapted to cause the control unit to control, in real time, the operation of the surgical tool in view of relative positions of the working end and of a given landmark indicative of the anatomical structure of interest.

In some implementations of the present technology, the computer is further adapted to compare, in real time, the path of the surgical tool with a path defined in a treatment plan corresponding to the surgical procedure, and evaluate, in real time, a compliance of the path of the surgical tool with the path defined in the treatment plan.

In some implementations of the present technology, the computer is further adapted to cause the control unit to stop operation of the surgical tool when the path of the surgical tool does not comply with the path defined in the treatment plan.

In some implementations of the present technology, the computer is further adapted to cause the control unit modify a trajectory of the surgical tool when the path of the surgical tool does not comply with the path defined in the treatment plan.

In some implementations of the present technology, the computer is further adapted to cause the control unit modify an operating speed of the surgical tool when the path of the surgical tool does not comply with the path defined in the treatment plan.

In some implementations of the present technology, the computer is further adapted to cause the display device to display a warning sign when the path of the surgical tool does not comply with the path defined in the treatment plan.

In some implementations of the present technology, the treatment plan includes a dental prosthetic plan and the path is defined in the treatment plan in view of improving at least one of a function and an appearance of a dental restoration.

In some implementations of the present technology, the treatment plan is based at least in part on an intraoral surface scan.

In some implementations of the present technology, the surgical tool comprises a drill, the anatomical structure of interest includes a mandible or a maxilla of a patient, and the dental prosthetic plan includes inserting an end of an implant in the mandible or the maxilla of the patient and mounting a prosthesis on an opposite end of the implant.

In some implementations of the present technology, the display device is a head-mountable display.

In some implementations of the present technology, the head-mountable display comprises a field-of-view (FOV) camera operatively connected to the computer and adapted to provide images of the field of view of the user to the computer, and the computer is adapted to create the augmented reality view further based on the images of the field of view of the user.

In some implementations of the present technology, the computer is further adapted to cause the display device to display the augmented reality view when the computer detects that the anatomical structure of interest is within the field of view of the user.

In some implementations of the present technology, the computer is further adapted to cause the display device to display a virtual reality view of the anatomical structure of interest when the computer detects that the anatomical structure of interest is not within the field of view of the user.

In some implementations of the present technology, the computer is further adapted to cause the display device to display a virtual reality view of a predicted outcome of the surgical procedure when the computer detects that the anatomical structure of interest is not within the field of view of the user.

In some implementations of the present technology, the computer is further adapted to predict, in real time, an outcome of the surgical procedure, and include a view of the predicted outcome of the surgical procedure in the augmented reality view.

In some implementations of the present technology, the system is used to assist an implantology procedure in dental surgery.

According to the present disclosure, there is also provided a method for assisting a user in a surgical procedure. Three dimensional (3D) positioning information about an anatomical structure of interest is acquired. 3D positioning information about a surgical tool is also acquired. A path of the surgical tool is determined. An augmented reality view of spatial relations between the anatomical structure of interest and one or more positions along the path of the surgical tool is created based on the 3D positioning information. The augmented reality view is displayed, superimposed over a field of view of the user.

In some implementations of the present technology, the surgical procedure is planned in view of improving at least one of a function and an appearance of a dental restoration.

In some implementations of the present technology, the path of the surgical tool is determined at least in part based on a current position of the surgical tool.

In some implementations of the present technology, the path of the surgical tool is further determined at least in part based on a previous position of the surgical tool.

In some implementations of the present technology, the path of the surgical tool is further determined at least in part based on a predicted position of the surgical tool calculated based on a current position of the surgical tool and on a current orientation of the surgical tool.

In some implementations of the present technology, acquiring the 3D positioning information about the anatomical structure of interest comprises acquiring 3D positioning information about a plurality of landmarks indicative of the anatomical structure of interest.

In some implementations of the present technology, the method further comprises positioning at least one fiducial marker proximally to the anatomical structure of interest, and acquiring 3D positioning information about the at least one fiducial marker, the augmented reality view being created further based on the 3D positioning information about the at least one fiducial marker.

In some implementations of the present technology, the at least one fiducial marker is an infrared emitter, acquiring 3D positioning information about the at least one fiducial marker comprising using an infrared detector.

In some implementations of the present technology, the at least one fiducial marker comprises a plurality of fiducial markers, the method further comprising triangulating the 3D positioning information about the plurality of markers.

In some implementations of the present technology, one of the at least one fiducial marker is a 3D fiducial structure, and acquiring 3D positioning information about the at least one fiducial marker comprises acquiring 3D positioning information about a plurality of reference points on the 3D fiducial structure.

In some implementations of the present technology, the method further comprises acquiring a 3D map of the anatomical structure of interest and of the at least one fiducial marker, the augmented reality view being created further based on the 3D map.

In some implementations of the present technology, the 3D map is acquired, before the surgical procedure, from an apparatus selected from a computerized tomography (CT) scanner, a cone beam computed tomography (CBCT) scanner and a magnetic resonance imaging (MRI) device, and the 3D map is stored in a database for access thereto during the surgical procedure.

In some implementations of the present technology, the method further comprises using a 3D camera to capture images of the anatomical structure of interest, of the at least one fiducial marker, and of the surgical tool, and performing a registration between images captured by the 3D camera and a content of the 3D map to update the 3D map.

In some implementations of the present technology, the method further comprises using a 3D camera to capture images of the anatomical structure of interest, of the at least one fiducial marker and of the surgical tool, and generating the 3D map based on the captured images.

In some implementations of the present technology, the method further comprises acquiring a 3D map of the anatomical structure of interest, the augmented reality view being created further based on the 3D map.

In some implementations of the present technology, the 3D map is acquired, before the surgical procedure, from an apparatus selected from a computerized tomography (CT) scanner, a cone beam computed tomography (CBCT) scanner and a magnetic resonance imaging (MRI) device, and the 3D map is stored in a database for access thereto during the surgical procedure.

In some implementations of the present technology, the method further comprises using a 3D camera to capture images of the anatomical structure of interest and of the surgical tool, and performing a registration between images captured by the 3D camera and a content of the 3D map to update the 3D map.

In some implementations of the present technology, the method further comprises using a 3D camera to capture images of the anatomical structure of interest and of the surgical tool, and generating the 3D map based on the captured images.

In some implementations of the present technology, the 3D map comprises a plurality of voxels distributed over three dimensions, each voxel having at least one intensity value and a coordinate over each of the three dimensions.

In some implementations of the present technology, each voxel has at least one polychromatic value, the at least one polychromatic value being derivable from the at least one intensity value.

In some implementations of the present technology, the 3D map comprises position, orientation and scale information of features of the anatomical structure of interest.

In some implementations of the present technology, the method further comprises selecting elements of the 3D map representing at least one cross-section of the anatomical structure of interest, the augmented reality view being created further based on the at least one cross-section of the anatomical structure of interest.

In some implementations of the present technology, the 3D positioning information about the about the anatomical structure of interest and the 3D positioning information about the surgical tool are acquired in real time, the method further comprising determining, in real time, spatial relations between the one or more positions along the path of the surgical tool and a position of a given landmark indicative of the anatomical structure of interest, and controlling, in real time, an operation of the surgical tool in view of the spatial relations between the one or more positions along the path of the surgical tool and the position of the given landmark indicative of the anatomical structure of interest.

In some implementations of the present technology, the surgical tool comprises a working end, the method further comprising acquiring, in real time, positioning information about the working end, determining, in real time, relative positions of the working end of the surgical tool and of a given landmark indicative of the anatomical structure of interest, and controlling, in real time, an operation of the surgical tool in view of the relative positions of the working end of the surgical tool and of the given landmark indicative of the anatomical structure of interest.

In some implementations of the present technology, the method further comprises tracking, in real time, a progression of the position of the surgical tool, comparing, in real time, the path of the surgical tool with a path defined in a treatment plan corresponding to the surgical procedure, and evaluating, in real time, a compliance of the path of the surgical tool with the path defined in the treatment plan.

In some implementations of the present technology, the method further comprises stopping operation of the surgical tool when the path of the surgical tool does not comply with the path defined in the treatment plan.

In some implementations of the present technology, the method further comprises modifying a trajectory of the surgical tool when the path of the surgical tool does not comply with the path defined in the treatment plan.

In some implementations of the present technology, the method further comprises modifying an operating speed of the surgical tool when the path of the surgical tool does not comply with the path defined in the treatment plan.

In some implementations of the present technology, the method further comprises displaying a warning sign when the path of the surgical tool does not comply with the path defined in the treatment plan.

In some implementations of the present technology, the treatment plan includes a dental prosthetic plan.

In some implementations of the present technology, the treatment plan is based at least in part on an intraoral surface scan.

In some implementations of the present technology, the surgical tool comprises a drill, the anatomical structure of interest includes a mandible or a maxilla of a patient, and the dental prosthetic plan includes inserting an end of an implant in the mandible or the maxilla of the patient and mounting a prosthesis on an opposite end of the implant.

In some implementations of the present technology, the method further comprises using a head-mountable display to display the augmented reality view superimposed over the field of view of the user.

In some implementations of the present technology, the method further comprises using a field-of-view (FOV) camera to acquire images of the field of view of the user, the augmented reality view being created further based on the images of the field of view of the user.

In some implementations of the present technology, the method further comprises displaying a predicted outcome of the surgical procedure as a part of the augmented reality view superimposed over the field of view of the user.

In some implementations of the present technology, the predicted outcome is calculated based on the path of the surgical tool.

In some implementations of the present technology, the method is used to assist an implantology procedure in dental surgery.

The foregoing and other features will become more apparent upon reading of the following non-restrictive description of illustrative embodiments thereof, given by way of example only with reference to the accompanying drawings.

### BRIEF DESCRIPTION OF THE DRAWINGS

Embodiments of the disclosure will be described by way of example only with reference to the accompanying drawings, in which:

FIG. 1 is a schematic block diagram showing components of a system for assisting a user in a surgical procedure according to an embodiment;

FIG. 2 is a schematic block diagram showing components of the computer of FIG. 1 according to an embodiment; and

FIGS. 3a, 3b and 3c are sequence diagrams showing operations of a method for assisting a user in a surgical procedure according to an embodiment.

Like numerals represent like features on the various drawings.

### DETAILED DESCRIPTION

Various aspects of the present disclosure generally address one or more of the problems related to the lack of details related to the position of surgical tools and to alterations of anatomical features in the course of a surgical procedure.

The present disclosure introduces an augmented reality system for use during surgical procedures including, but not limited to, implantology in dental surgery. One example of use of the present disclosure is in the course of a typical dental surgical procedure to position an implant-supported tooth prosthesis, for example a crown, in the mouth of a patient.

A clinician may prepare a treatment plan before initiating a surgical procedure. Continuing with the example of use that relates to a dental surgical procedure, the treatment plan may for example define a planned implant position with respect to a desired tooth prosthesis position and to the underlying bone. Although the treatment plan is conventionally prepared in advance of the surgical procedure to define a desired implant position with respect to anatomical structures using detailed information from a medical imaging system, the final position of the implant cannot be verified until after the surgical procedure through additional use of the medical imaging system. During the surgical procedure, the clinician is unable to see the position of a surgical tool, for example a distal end of a drill bit, as the drill bit penetrates a bone. The clinician is therefore unable to verify (1) a proximity of the drill bit to important anatomical structures such as nerves, blood vessels and other teeth, and (2) whether the drill bit follows a correct path in accordance the treatment plan. Of course, precise and controlled operation of any surgical tool is important in any surgical procedure. However, in many cases of restorative surgical procedures, functional and/or aesthetic aspects are also critical, as the position and shape of an implant-supported tooth prosthesis, such as a crown, may have an impact on the function and/or appearance of a dental restoration and on the satisfaction and/or well-being of the patient.

The present disclosure provides a three-dimensional (3D) augmented reality image of an underlying anatomical structure of interest of the patient, for example teeth roots, maxilla, mandible, inferior alveolar nerve, and the like, with the ability to see images showing a depth profile of the underlying anatomical structure and its sub-structures, for example as in a cross-sectional view. The augmented reality image of the anatomical structures and sub-structures can be updated in real time as the anatomical structure is penetrated, for example during cutting or drilling into the bone. This allows the clinician to accurately position an implant in the bone during a surgical procedure whilst avoiding contact with certain anatomical substructures such as nerves and blood vessels.

The system could be used in conjunction with the technology described in U.S. Pat. No. 9,179,843, the contents of which are incorporated herein by reference, to avoid contact with certain anatomical substructures.

Still continuing with the example of use that relates to a dental surgical procedure, the treatment plan, which may define the implant dimensions and/or the implant position with respect to the underlying bone, to soft tissue surrounding thereof, to other implant positions and to a desired dental prosthesis position and structure, can be included in the 3D augmented reality image, allowing the clinician to view in real time a desired drill path, including a position, orientation and depth of the drill bit. The planned implant position may be represented as a contour and/or as a longitudinal axis of the implant within the underlying bone. An illustration based on the treatment plan may be included in the 3D augmented reality image, allowing a real time evaluation of a path actually being taken by a surgical tool, such as a drill, in comparison with a planned path of the tool corresponding to the planned implant position.

The system may also update the treatment plan based on changes to the underlying bone, to the surrounding soft tissue, to other implant positions and to the desired tooth prosthesis position and structure when such changes occur in the course of the surgical procedure. As the clinician drills into the underlying bone of the patient, the actual drill path is tracked. If the system detects that the actual drill path is off course in relation to the treatment plan, the system may control the operation of the surgical tool by reducing, modifying, or stopping any action of the drill. The system may trigger an alarm to warn the clinician, or provide drill path correction instructions. The system may also display information indicative of expected outcomes of the surgical procedures. In particular, the system may include a live update of a predicted outcome of the surgical procedure in the augmented reality image—the image may in such case be understood as a virtual reality image because the predicted outcome being shown is a virtual entity that is not yet present within the anatomical structure of interest. It is contemplated that desired and predicted positions of an implant may be displayed alternately and/or concurrently within the augmented reality image. This allows the clinician to correct the drill path to stay true to treatment plan or to address complications arising during the surgical procedure. Furthermore, the clinician is assisted by the system in avoiding undesirable contact with certain anatomical structures such as nerves, blood vessels, teeth, and the like, while being assisted in achieving desirable surgical and prosthetic outcomes.

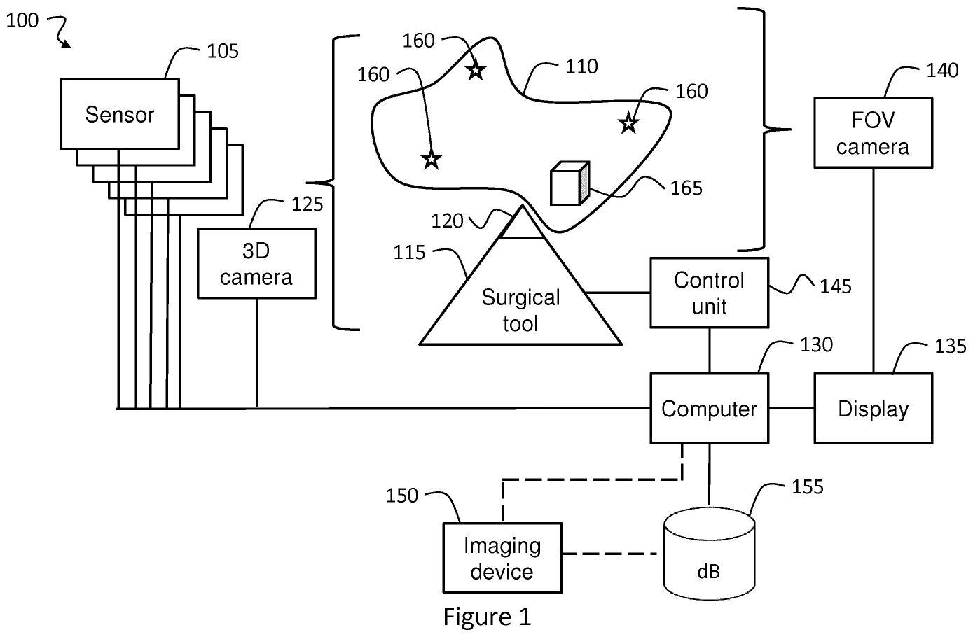

Referring now to the drawings, FIG. 1 is a schematic block diagram showing components of a system for assisting a user in a surgical procedure according to an embodiment. In the embodiment of FIG. 1, a system 100 includes sensors 105 positioned in view of an anatomical structure of interest 110 and of a surgical tool 115 having a working end 120. The sensors 105 may include a 3D camera 125. Various types of sensors 105 may be used, for example and without limitation a proximity sensor, an optical sensor, a spectral absorption sensor, an accelerometer or a gyroscope. The anatomical structure of interest 110 may be any part of a patient's anatomy on which a clinician will perform the surgical procedure. The system 100 also includes a computer 130, a display device 135, a field-of-view (FOV) camera 140, a control unit 145, a medical imaging device 150 and a database (dB) 155. The computer 130 may store in a memory a treatment plan for the surgical procedure. The display device 135 and the FOV camera 140 may be integrated in a head-mountable display wearable by a user, for example a dental or medical clinician. The FOV camera 140 may provide the computer 130 with images in a field of view of the user, images of the field of view being captured by the FOV camera 140 and provided to the computer 130. Fiducial markers 160 may be placed on various areas of the anatomical structure of interest 110. One of the fiducial markers 160 may be a fiducial structure 165 having a 3D structure on which several reference points may be defined. In a variant, the fiducial markers 160 may include infrared emitters (not shown) and at least one of the sensors 105 may be capable of detecting infrared signals emitted by the fiducial markers. Use of fiducial markers 160 capable of passively reflecting infrared light emitting from an infrared light source (not shown) is also contemplated.

Some of the components 105-165 of the system 100 are optional and may not be included in other embodiments. The control unit 145 may be implemented as a software module in the computer 130 or as a separate hardware module.

The sensors 105, including the 3D camera 125 when present, provide 3D positioning information about the anatomical structure of interest 110 to the computer 130, and may provide 3D positioning information about specific landmarks of the anatomical structure of interest 110. The sensors 105 also provide 3D positioning information about the surgical tool 115 to the computer 130, for example about the working end 120 of the surgical tool 115. In a variant, one of the sensors 105 may be mounted on the working end 120 of the surgical tool 115. The sensors 105 may further provide 3D positioning information about the fiducial markers 160 when these are positioned on the anatomical structure of interest 110. At least one of the sensors 105 may be capable of providing 3D positioning information about reference points on the structure 165 to the computer 130. Information from at least one of the sensors 105 and/or from the 3D camera 125 may be provided in real time. The computer 130 may triangulate the 3D positioning information about the fiducial markers 160 to form at least in part a 3D map of the anatomical structure of interest 110. It should be understood that, throughout the present disclosure, the expression “3D positioning information” includes inter alia an orientation of the object of this positioning information, whether this object is the anatomical structure of interest 110 and/or its landmarks, the surgical tool 115 and/or its working end 120, the fiducial markers 160, the fiducial structure 165 and/or its reference points.

The control unit 145 may relay commands from the computer 130 to control at least in part an operation of the surgical tool 115.

The medical imaging device 150, when present, may include one or more of a computerized tomography (CT) scanner, a cone beam computed tomography (CBCT) scanner, and a magnetic resonance imaging (MRI) device. Alternatively or in addition, the medical imaging device 150 may provide a plain two-dimensional X-ray image that may be supplemented with other imaging modalities to add depth information, for example by using a spectral absorption probe as described in U.S. Pat. No. 9,179,843 B2, issued on Nov. 10, 2015, the disclosure of which is incorporated by reference in its entirety. The medical imaging device 150 prepares a 3D map of the anatomical structure of interest 110 and of the fiducial markers 160. Complementary datasets, defining intraoral surface scans (for dentistry applications), prosthetic plans, and the like, may be registered to existing datasets to form 2D and/or 3D coordinates and geometry that made part of the 3D map. The 3D map may be provided by the medical imaging device 150 directly to the dB 155 for storage. Alternatively, the 3D map may be provided to the computer 130 that in turn stores the 3D map in the dB 155 for later retrieval.

The 3D camera 125, when present, captures images of the anatomical structure of interest 110, of the at least one fiducial marker 160, and of the surgical tool 115 and provides these images to the computer 130. The computer 130 may perform a registration between the images captured by the 3D camera 125 and a content of the 3D map to update the 3D map. The image capture and the registration may be performed in real time. Alternatively, the computer 130 may generate the 3D map on the basis of the captured images.

In an embodiment, the 3D map comprises a plurality of voxels distributed over three dimensions. Each voxel has at least one intensity value and a coordinate over each of the three dimensions. Each voxel may have at least one polychromatic value, the at least one polychromatic value being derivable from the at least one intensity value. The 3D map may contain position, orientation and scale information of features of the anatomical structure of interest 110.

In operation, the computer 130 uses the 3D positioning information provided by the sensors 105 to determine a path of the surgical tool 115, or of its working end 120. More specifically, the path is based at least in part on a current position of the surgical tool 115 or of its working end 120, on one or more previous positions of the surgical tool 115 or of its working end 120 and/or a predicted position of the surgical tool 115 or of its working end 120 calculated based on a current position of the surgical tool 115 or of its working end 120 and on a current orientation of the surgical tool 115 or of its working end 120. The computer 130 then creates an augmented reality view of spatial relations between the anatomical structure of interest 110 and one or more positions along the path of the surgical tool 115 or of its working end 120 based on the 3D positioning information. The augmented reality view may further be created by the computer 130 on the basis of the 3D positioning information about the fiducial markers, on the basis of the 3D map, and/or on the basis of the images in the field of view of the user. In an embodiment, the computer 130 may select elements of the 3D map representing one or more cross-sections of the anatomical structure of interest 110. The selection of the elements that are part of the one or more cross-sections may be based on a treatment plan for the surgical procedure. The augmented reality view may further be created, by the computer, on the basis of the one or more cross-sections of the anatomical structure of interest 110. The computer 130 may create the augmented reality view in real time. At least one of the sensors 105 may provide, in real time, positioning and/or depth information of the working end 120 of the surgical tool 115 with respect to a landmark of the anatomical structure of interest 110. The positioning and/or depth information may be added to complement the 3D map and the augmented reality view.

Having created the augmented reality view, the computer 130 then causes the display device 135 to display the augmented reality view superimposed over the field of view of the user. In a variant, the display device 135 may comprise a transparent screen on which the augmented reality view can be displayed while allowing the user to normally see what is in her field of view. In another variant, the display device 135 may comprise an opaque screen on which the augmented reality view as well as a camera-image of the field of view of the user captured by the FOV camera 140 can be displayed. Holographic projection of the augmented reality view over the field of view of the user, i.e. over the anatomical structure of interest 110, is also contemplated.

In an embodiment, the computer 130 may be capable to detect, based on an image received from the FOV camera 140, whether or not the anatomical structure of interest 110 is within the field of view of the user. The computer 130 may then cause the display device 135 to display the augmented reality view on the condition that the anatomical structure of interest 110 is in fact within the field of view of the user. In the same or another embodiment, the computer 130 may cause the display device 135 to display a virtual reality view of the anatomical structure of interest 110 when the computer 130 detects that the anatomical structure of interest 110 is not within the field of view of the user. In the same or yet another embodiment, the computer 130 may cause the display device 135 to display a virtual reality view of a predicted outcome of the surgical procedure when the computer 130 detects that the anatomical structure of interest 110 is not within the field of view of the user.

Still in the same or another embodiment, the system 100 may control, in real time, an operation of the surgical tool 115. To this end, the computer 130 determines a path taken by the surgical tool 115, or of its working end 120, and evaluates spatial relations between one or more positions along this path and a position of a given landmark indicative of the anatomical structure of interest 110 to the control unit 145. Possible landmarks of the anatomical structure of interest 110 may comprise an indication of a bone density, a nerve, a tooth, a blood vessel, and the like. The control unit 145 controls the operation of the surgical tool 115 in view of the spatial relations between the surgical tool 115, or its working end 120, and the landmark indicative of the anatomical structure of interest 110.

The computer 130 may track, in real time, the path taken by the surgical tool 115, or of its working end 120, in the course of the surgical procedure, and evaluate, in real time, a compliance of the path taken by the surgical tool 115, or by its working end 120, with a path defined in the treatment plan. The computer 130 may cause the control unit 145 to control, in real-time, an operation of the surgical tool 115 in view of this evaluation. Following a non-compliance detection, the computer 130 may cause the control unit 145 to stop operation of the surgical tool 115, modify an operating speed of the surgical tool 115, modify a trajectory of the surgical tool by modifying its axis and/or cause the display device 135 to display a warning sign, a drill path correction instruction or an information indicative of a surgical or prosthetic outcome. Whether or not the path taken by the surgical tool 115, or by its working end 120, complies with the path defined the treatment plan, the computer 130 may predict an outcome of the surgical procedure and cause the display 135 to display this predicted outcome as a part of the augmented reality view visible over the field of view of the user.

In a non-limiting example, the system 100 may be used to assist an implantology procedure in dental surgery. To this end, some fiducial markers 160 may be positioned with respect to or proximally to gums and/or teeth of a patient. The treatment plan may be based at least in part on an intraoral surface scan and may include a dental prosthetic plan that, in turn, may include inserting an end of an implant in the mandible or maxilla of the patient and mounting a prosthesis, such as a crown, on an opposite end of the implant. The path of the surgical tool 115 may be defined in the treatment plan in view of improving a function and/or an appearance of a dental restoration. The surgical tool 115 may be a drill or a saw and the working end of the tool 120 may be a tip of a drill bit or a blade of the saw. The system 100 may for example assist a dental surgeon in drilling into a maxillary or mandibular bone of the patient in the preparation of inserting an implant. Contact of the drill bit with some landmarks of the anatomical structure of interest 110, for example a nerve, a tooth or a blood vessel, may need be avoided. Based on a pre-planned 3D position of the implant in the maxillary or mandibular bone of the patient, the computer 130 may detect that the drill bit is in an incorrect position and cause the control unit 145 to modify a path of the drill bit.

Of course, dental implantology is only one of possible uses of the system 100 and the present disclosure is not so limited.

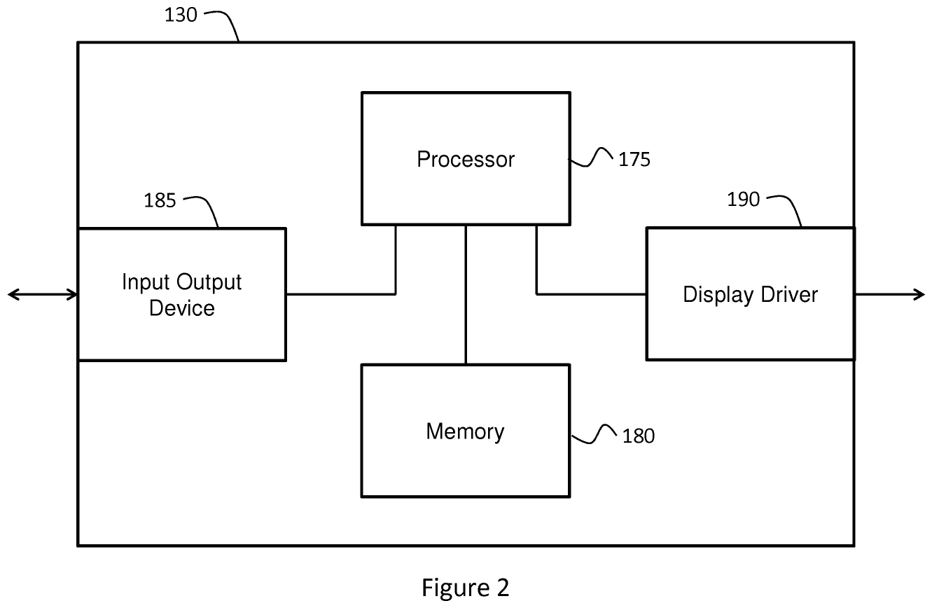

FIG. 2 is a schematic block diagram showing components of the computer of FIG. 1 according to an embodiment. The computer 130 includes a processor 175, a memory 180, an input-output device 185 and a display driver 190. The processor 175 is operatively connected to the memory 180, to the input-output device 185, and to the display driver 190 of the computer 130.

In the computer 130, the processor 175 uses the input-output device 180 to communicate with the sensors 105, the 3D camera 125, the FOV camera 140, the control unit 145, the imaging device 150 and the dB 155 when performing the functions of the computer 130 as described in the foregoing description of FIG. 1. The processor acquires the positioning information received from the sensors 105, from the 3D camera 125 and from the FOV camera 140. The processor 175 reads the 3D map from the dB 155 and/or obtains information allowing constructing the 3D map directly from the medical imaging device 150. The processor 175 uses these information elements to create the augmented reality view. The processor 175 causes the display driver 190 to format the augmented reality view for use by the display 130.

The memory 180 stores various parameters related to the operation of the system 100 including, without limitation, a treatment plan for a patient. The memory 180 may also store, at least temporarily, some of the information acquired at the computer 130 from the sensors 105, the 3D camera 125, and the FOV camera 140, as well as from the imaging device 150 and/or the dB 155.

The memory 180 may further store non-transitory executable code that, when executed by the processor 175, cause the processor 175 to implement the various functions of the computer 130 described in the foregoing description of FIG. 1.

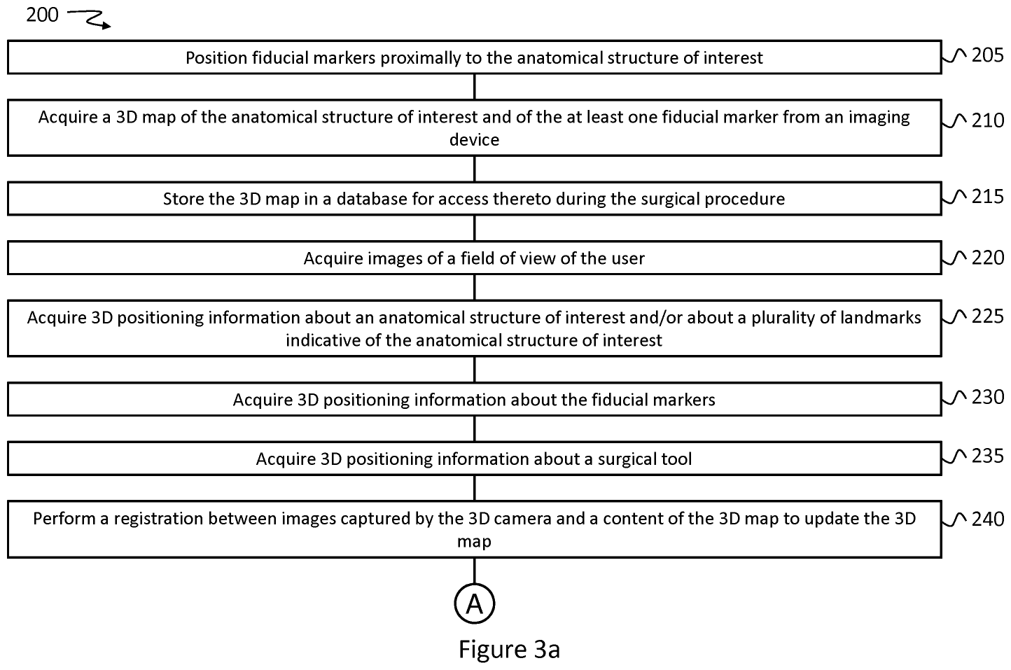

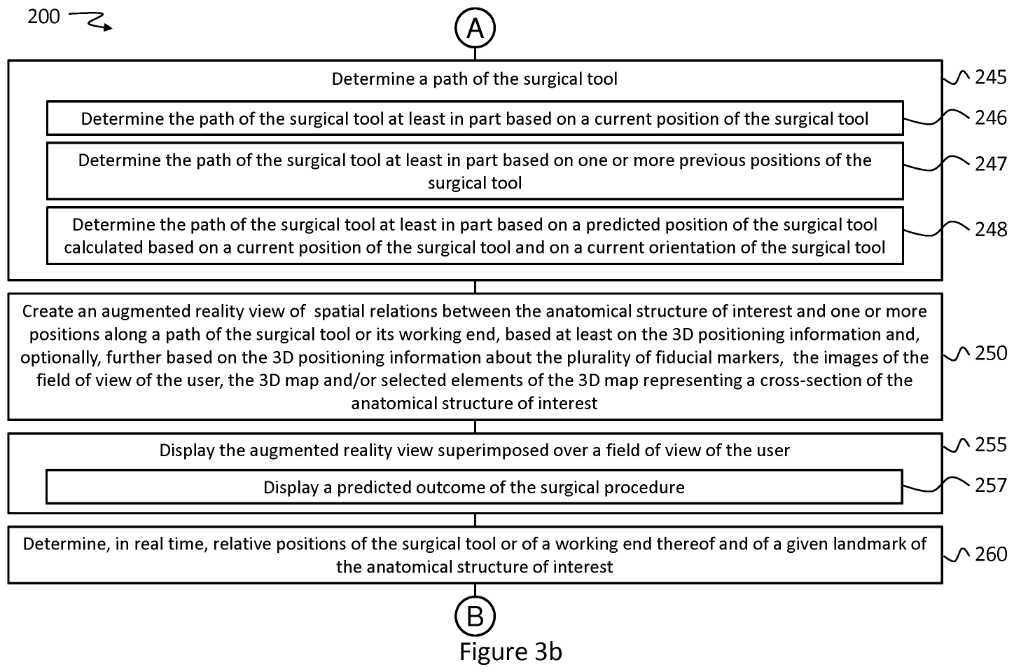

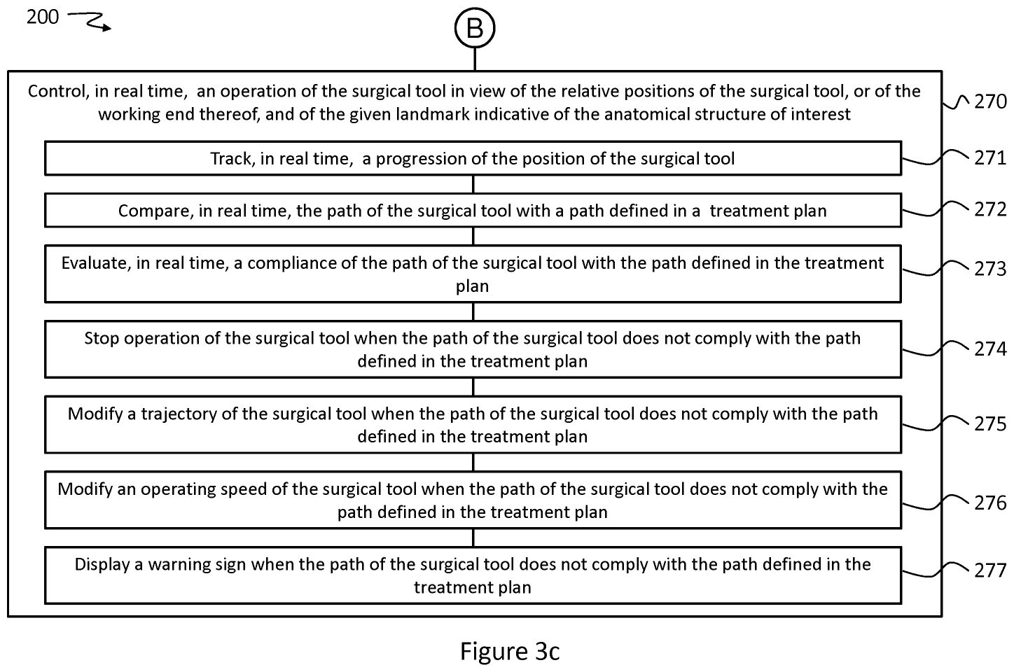

FIGS. 3a, 3b and 3c are a sequence diagram showing operations of a method for assisting a user in a surgical procedure according to an embodiment. In FIGS. 3a, 3b and 3c, a sequence 200 comprises a plurality of operations, some of which may be executed in variable order, some of the operations possibly being executed concurrently, some of the operations being optional.

An optional pre-surgical phase may include some of the following operations:

Operation 205: One or more fiducial markers 150 are positioned proximally to the anatomical structure of interest 110. Among the fiducial makers 160 may be one or more 3D fiducial structure 165 on which several reference points may be defined.

Operation 210: A 3D map of the anatomical structure of interest 110 and, optionally, of the fiducial markers 160, is acquired. The 3D map may be supplied from the medical imaging device 150, for example a CT scanner, a CBCT scanner, or an MRI device. Alternatively, the 3D camera 125 may be used to capture images of the anatomical structure of interest 110 and, optionally, of the fiducial markers 110. The 3D map may be generated based on the captured images. In some embodiments, the 3D map comprises a plurality of voxels distributed over three dimensions, each voxel having at least one intensity value and a coordinate over each of the three dimensions. Each voxel may have at least one polychromatic value, the at least one polychromatic value being derivable from the at least one intensity value. In the same or other embodiments, the 3D map may comprise position, orientation and scale information of features of the anatomical structure of interest 110.

Operation 215: The 3D map is stored in the dB 155 for access thereto during the surgical procedure.

A surgical assistance phase may include all or a subset of the following operations:

Operation 220: Images of the field of view of the user may be acquired from the FOV camera 140. The images of the field of view of the user may be acquired in real time.

Operation 225: 3D positioning information about the anatomical structure of interest 110 is acquired. This may include acquiring 3D positioning information about various landmarks indicative of the anatomical structure of interest 110. The 3D positioning information about the anatomical structure of interest 110 may be acquired in real time.

Operation 230: 3D positioning information about the fiducial markers 160 is acquired, if such fiducial markers are placed on the anatomical structure of interest 110. For example, when a fiducial marker 160 is an infrared emitter, one of the sensors 105 may comprise an infrared detector. When one of the fiducial markers 160 is the fiducial structure 165, 3D positioning information may be acquired about the reference points on the 3D fiducial structure. Triangulation of the 3D positioning information about several markers 160 may be performed. The 3D positioning information about the fiducial markers 160 may be acquired in real time.

Operation 235: 3D positioning information about the surgical tool 115, optionally about the working end 120, is acquired. The 3D positioning information about the surgical tool 115, or about the working end 120 may be acquired in real time.

Operation 240: A registration may be made between the images captured by the 3D camera 125 and a content of the 3D map to update the 3D map. The registration may be made in real time.

Operation 245: A path of the surgical tool 115 or a path of its working end 120 is determined. This operation may comprise one or more sub-operations 246, 247 and 248.

Sub-operation 246: The path of the surgical tool 115 or of its working end 120 may be determined at least in part based on a current position of the surgical tool 115 or of its working end 120.

Sub-operation 247: The path of the surgical tool 115 or of its working end 120 may be determined at least in part based on one or more previous positions of the surgical tool 115 or of its working end 120.

Sub-operation 248: The path of the surgical tool 115 or of its working end 120 may be determined at least in part based on a predicted position of the surgical tool 115 or of its working end 120 calculated based on a current position of the surgical tool 115 or of its working end 120 and on a current orientation of the surgical tool 115 or of its working end 120.

Operation 250: An augmented reality view is created based on the 3D positioning information. The augmented reality view represents spatial relations between the anatomical structure of interest 110 and one or more positions along the path of the surgical tool 115 or the path of its working end 120. Additional information elements may be used to create the augmented reality view. Without limitation, the augmented reality view may further be created based on the images of the field of view of the user, on the 3D positioning information about the fiducial markers 160, and/or based on the 3D map. For instance, elements of the 3D map representing one or more cross-sections of the anatomical structure of interest 110 may be selected, the augmented reality view being further based on the one or more cross-sections of the anatomical structure of interest 110.

Operation 255: The augmented reality view is displayed superimposed over the field of view of the user. In an embodiment, a head-mountable display combining the display device 135 and the FOV camera 140 may be used to display the augmented reality view. The augmented reality view may be displayed in real time. Operation 255 may include sub-operation 257.

Sub-operation 257: A predicted outcome of the surgical procedure may be displayed as a part of the augmented reality view visible over the field of view of the user. In a non-limiting example where the surgical procedure is made in view of inserting an implant in the anatomical structure of interest 110, the augmented reality view may show an expected position of the implant based on the path of the surgical tool. Displaying at once the expected position of the implant and a desired position of the implant as defined in a treatment plan stored in the computer 130 is also contemplated.

Operation 260: Relative positions of the surgical tool 115, or of its working end 120, and of a given landmark of the anatomical structure of interest 110 are determined in real time.

Operation 270: An operation of the surgical tool 115 may be controlled in real time at least in part in view of the relative positions of the surgical tool 115, or of its working end 120, and of the given landmark indicative of the anatomical structure of interest 110. This operation may comprise one or more of sub-operations 271 to 277.

Sub-operation 271: A progression of the position of the surgical tool 115, or of its working end 120, may be tracked in real time.

Sub-operation 272: The path of the surgical tool 115, or of its working end 120, may be compared in real time with a path defined in the treatment plan.

Sub-operation 273: A compliance of the path of the surgical tool or of its working end with the path defined in the treatment plan is evaluated in real time.

Sub-operation 274: Operation of the surgical tool 115 may be stopped when the path of the surgical tool does not comply with the path defined in the treatment plan.

Sub-operation 275: A trajectory of the surgical tool 115 may be modified when the path of the surgical tool does not comply with the path defined in the treatment plan. This modification of the trajectory of the surgical tool 115 may be made in view of reducing a distance between the path of the surgical tool and the path defined in the treatment plan. In particular, this sub-operation may prevent cutting or drilling into a nerve or into a blood vessel.

Sub-operation 276: An operating speed of the surgical tool 115 may be modified when the path of the surgical tool does not comply with the path defined in the treatment plan. This may be effective, for example, in preventing overheating of a bone being cut or perforated by the surgical tool 115.

Sub-operation 277: A warning sign may be displayed, for example on the display device 135, when the path of the surgical tool does not comply with the path defined in the treatment plan.

The method illustrated in the sequence 200 may be applied in various procedures, including without limitation to assist an implantology procedure in dental surgery. As such, the surgical procedure may be planned in view of improving a function and/or an appearance of a dental restoration. The treatment plan may include a dental prosthetic plan that may, in turn, be based at least in part on an intraoral surface scan. To install a dental prosthesis in the mouth of a patient, the surgical tool 115 may comprise a drill and its working end 120 may comprise the tip of a drill bit. The anatomical structure of interest 110 may comprise a mandible or a maxilla of the patient. The dental prosthetic plan may include inserting one end of an implant, for example a screw, in the mandible or the maxilla of the patient, and mounting the prosthesis, for example a crown, on an opposite end of the implant.

Each of the operations of the sequence 200 may be configured to be processed by one or more processors, the one or more processors being coupled to a memory, for example and without limitation the processor 175 and the memory 180 of FIG. 2.

Various embodiments of the system and method for assisting a user in a surgical procedure, as disclosed herein, may be envisioned. The following paragraphs describe implementation examples of the present system and method and may include various optional features that may be present in some embodiments and not in other embodiments.

The present technology may be considered in view of three (3) phases of a surgical process, including (1) a data acquisition phase, (2) a pre-operatory phase, and (3) a per-operatory phase.

### Acquisition of Data

A medical imaging device is used to perform data acquisition of a patient's anatomy of interest. The imaging modality used (CT, CBCT, MRI, etc.) produces a volumetric representation of the full internal structure of the anatomy (i.e. medical dataset). The resulting medical dataset is usually tomographic (3D volume, discretized in 2D slices that are defined perpendicularly to a default orientation). A reference spatial relationship is defined between the anatomy and a fiducial structure. The imaging device generates a medical dataset of the anatomy and of the fiducial structure when in the reference spatial relationship. The medical dataset is exported from the imaging device to a computer for further processing and use, using dedicated software.

### Pre-Operatory Processing and Use of Data

Pre-processing of the medical dataset is performed, for example using a field of view restriction, an orientation correction and/or filtering of the medical dataset to yield a processed medical dataset. Clinically-relevant geometry is identified and/or defined, highlighting for example a panoramic curve, anatomical landmarks & tooth sites. As a result, 2D and/or 3D coordinates and geometry and at least parts of a treatment plan dataset are defined.

Anatomical structures, for example inferior alveolar nerve, mandible and/or maxilla, teeth, existing implants, are segmented from the medical dataset. Without limitation, segmentation may be performed slice by slice. An approximation of the contours of each structure, usually defined as coordinates (x,y) in each slice (z) in which the structure appears is obtained.

Segmented portions are reconstructed, resulting in 3D surface datasets (i.e. meshes or shells) derived from the contour coordinates, forming at least another part of the treatment plan dataset.

Complementary datasets, defining intraoral surface scans, prosthetic plan, and the like, may be registered to existing datasets to form 2D and/or 3D coordinates and geometry that are added to the treatment plan dataset.

The position, orientation and dimensions are determined for implant receptor sites and/or osteotomy pathways. Implant size, depth and 3D angulation position are chosen. 3D representations of implants and surgical tools are defined based on position and orientation criteria for any subset of the treatment plan dataset. Resulting 2D and/or 3D coordinates and geometry are added to the treatment plan dataset.

### Per-Operatory Processing and Use of Data for Augmented Reality

At the onset of surgical procedure, an augmented reality system and surgical tools are provided in the clinical environment where the patient and the clinician are also present. The augmented reality system at least includes sensors, a computer, a controller and a display device. The reference spatial relationship between the anatomy and the fiducial structure is reproduced. The clinician and surgical tools have fiducial markers so that the sensors can follow their position.

During the surgical procedure, the sensors and computer are used to dynamically generate a position and orientation tracking dataset of the patient, optionally using a fiducial structure, and generate a position and orientation tracking dataset of the clinician and of the surgical tools, optionally using the fiducial markers.

A video dataset of the clinical environment is generated. A spatial relationship between the medical dataset, the tracking dataset and the video dataset is calculated. Images are generated, derived from the datasets and their spatial relationship. A computer controls image display parameters to include or exclude portions of the datasets, for example to include a cross-section of the medical dataset, generated according to a cross-section plane derived from the treatment plan dataset. The computer also controls display parameters to render image properties, for example transparency of 3D geometry, grayscale thresholds of medical dataset, and the like. The computer may also control surgical tool operation parameters, either directly or through a control unit. The control unit may be operated either automatically, for example according to spatial relationships and fulfillment of criteria from the treatment plan dataset, or interactively, by the clinician.

The display device is used to dynamically show the images. The display device may be integrated in a head-mountable display that, when worn by the clinician, allows to display the images in the field of view of the clinician. Such a head-mountable display is capable of showing images of the clinical environment derived from the video dataset such that the clinician's visualization of the clinical environment is not hindered by the display device, and is capable of showing images derived from the medical and treatment plan datasets such that the clinician's visualization of the clinical environment is augmented with information that may assist decision-making and risk management during the surgical procedure. In particular, the images may be shown in real time on the display device to allow visualizing a live update of a predicted outcome of the surgical procedure.

Those of ordinary skill in the art will realize that the description of the system and method for assisting a user in a surgical procedure are illustrative only and are not intended to be in any way limiting. Other embodiments will readily suggest themselves to such persons with ordinary skill in the art having the benefit of the present disclosure. Furthermore, the disclosed system and method may be customized to offer valuable solutions to existing needs and problems related to the lack of details related to the position of surgical tools and to alterations of anatomical features in the course of a surgical procedure. In the interest of clarity, not all of the routine features of the implementations of the system and method are shown and described. In particular, combinations of features are not limited to those presented in the foregoing description as combinations of elements listed in the appended claims form an integral part of the present disclosure. It will, of course, be appreciated that in the development of any such actual implementation of the system and method, numerous implementation-specific decisions may need to be made in order to achieve the developer's specific goals, such as compliance with application-related, system-related, network-related, and business-related constraints, and that these specific goals will vary from one implementation to another and from one developer to another. Moreover, it will be appreciated that a development effort might be complex and time-consuming, but would nevertheless be a routine undertaking of engineering for those of ordinary skill in the field of medical imaging having the benefit of the present disclosure.

In accordance with the present disclosure, the components, process operations, and/or data structures described herein may be implemented using various types of operating systems, computing platforms, network devices, computer programs, and/or general purpose machines. In addition, those of ordinary skill in the art will recognize that devices of a less general purpose nature, such as hardwired devices, field programmable gate arrays (FPGAs), application specific integrated circuits (ASICs), or the like, may also be used. Where a method comprising a series of operations is implemented by a computer, a processor operatively connected to a memory, or a machine, those operations may be stored as a series of instructions readable by the machine, processor or computer, and may be stored on a non-transitory, tangible medium.

Systems and modules described herein may comprise software, firmware, hardware, or any combination(s) of software, firmware, or hardware suitable for the purposes described herein. Software and other modules may be executed by a processor and reside on a memory of servers, workstations, personal computers, computerized tablets, personal digital assistants (PDA), and other devices suitable for the purposes described herein. Software and other modules may be accessible via local memory, via a network, via a browser or other application or via other means suitable for the purposes described herein. Data structures described herein may comprise computer files, variables, programming arrays, programming structures, or any electronic information storage schemes or methods, or any combinations thereof, suitable for the purposes described herein.

The present disclosure has been described in the foregoing specification by means of non-restrictive illustrative embodiments provided as examples. These illustrative embodiments may be modified at will. The scope of the claims should not be limited by the embodiments set forth in the examples, but should be given the broadest interpretation consistent with the description as a whole.

What is claimed is:

1. A system for assisting a user in a surgical procedure, comprising:
at least one sensor adapted to provide three dimensional (3D) positioning information about an anatomical structure of interest and about a surgical tool;
a computer operatively connected to the at least one sensor and adapted to:
determine a path of the surgical tool, and
create, based on the 3D positioning information, an augmented reality view of spatial relations between the anatomical structure of interest and one or more positions along the path of the surgical tool; and
a display device adapted to display the augmented reality view superimposed over a field of view of the user.

2. The system of claim 1, wherein the computer is further adapted to determine the path of the surgical tool at least in part based on a current position of the surgical tool.

3. The system of claim 1 or 2, wherein the computer is further adapted to determine the path of the surgical tool at least in part based on a previous position of the surgical tool.

4. The system of any one of claims 1 to 3, wherein the computer is further adapted to determine the path of the surgical tool at least in part based on a predicted position of the surgical tool calculated based on a current position of the surgical tool and on a current orientation of the surgical tool.

5. The system of any one of claims 1 to 4, wherein the at least one sensor is further adapted to provide 3D positioning information about a plurality of landmarks indicative of the anatomical structure of interest.

6. The system of any one of claims 1 to 5, further comprising:
at least one fiducial marker adapted for placement proximally to the anatomical structure of interest;
wherein:
the at least one sensor is further adapted to provide 3D positioning information about the at least one fiducial marker; and
the computer is adapted to create the augmented reality view further based on the 3D positioning information about the at least one fiducial marker.

7. The system of claim 6, wherein the at least one fiducial marker is an infrared emitter and the at least one sensor comprises an infrared detector.

8. The system of claim 6 or 7, wherein:
the at least one fiducial marker comprises a plurality of fiducial markers; and
the computer is further adapted to triangulate the 3D positioning information about the plurality of markers.

9. The system of any one of claims 6 to 8, wherein:
one of the at least one fiducial marker is a 3D fiducial structure; and
the at least one sensor is further adapted to provide 3D positioning information about a plurality of reference points on the 3D fiducial structure.

10. The system of any one of claims 6 to 9, further comprising:
a database operatively connected to the computer and storing a 3D map of the anatomical structure of interest and of the at least one fiducial marker;
wherein the computer is further adapted to create the augmented reality view based on the 3D map.

11. The system of claim 10, wherein the 3D map is obtained from an apparatus selected from one or more of: a computerized tomography (CT) scanner, a cone beam computed tomography (CBCT) scanner and a magnetic resonance imaging (MRI) device.

12. The system of claim 11, wherein:
the at least one sensor comprises a 3D camera adapted to capture images of the anatomical structure of interest, of the at least one fiducial marker, and of the surgical tool; and
the computer is further adapted to perform a registration between images captured by the 3D camera and a content of the 3D map to update the 3D map.

13. The system of claim 10, wherein:
the at least one sensor comprises a 3D camera adapted to capture images of the anatomical structure of interest, of the at least one fiducial marker, and of the surgical tool; and
the computer is further adapted to generate the 3D map based on the captured images.

14. The system of any one of claims 1 to 9, further comprising:
a database operatively connected to the computer and storing a 3D map of the anatomical structure of interest;
wherein the computer is further adapted to create the augmented reality view based on the 3D map.

15. The system of claim 14, wherein the 3D map is obtained from an apparatus selected from one or more of a computerized tomography (CT) scanner, a cone beam computed tomography (CBCT) scanner and a magnetic resonance imaging (MRI) device.

16. The system of claim 15, wherein:
the at least one sensor comprises a 3D camera adapted to capture images of the anatomical structure of interest and of the surgical tool; and
the computer is further adapted to perform a registration between images captured by the 3D camera and a content of the 3D map to update the 3D map.

17. The system of claim 16, wherein:
the at least one sensor comprises a 3D camera adapted to capture images of the anatomical structure of interest and of the surgical tool; and
the computer is further adapted to generate the 3D map based on the captured images.

18. The system of any one of claims 10 to 17, wherein the 3D map comprises a plurality of voxels distributed over three dimensions, each voxel having at least one intensity value and a coordinate over each of the three dimensions.

19. The system of claim 18, wherein each voxel has at least one polychromatic value, the at least one polychromatic value being derivable from the at least one intensity value.

20. The system of any one of claims 10 to 19, wherein the 3D map comprises position, orientation and scale information of features of the anatomical structure of interest.

21. The system of any one of claims 10 to 20, wherein the computer is further adapted to:
select elements of the 3D map representing at least one cross-section of the anatomical structure of interest; and
create the augmented reality view further based on the at least one cross-section of the anatomical structure of interest.

22. The system of any one of claims 1 to 21, further comprising a control unit operatively connected to the computer and adapted to control an operation of the surgical tool.

23. The system of claim 22, wherein:
the at least one sensor is further adapted to provide, in real time, the 3D positioning information about the anatomical structure of interest and about the surgical tool;
the computer is further adapted to provide, in real time, spatial relations between the one or more positions along the path of the surgical tool and a position of a given landmark indicative of the anatomical structure of interest to the control unit; and
the control unit is further adapted to control, in real time, the operation of the surgical tool in view of the spatial relations between the one or more positions along the path of the surgical tool and the position of the given landmark indicative of the anatomical structure of interest.

24. The system of claim 23, wherein:
the surgical tool comprises a working end;
the at least one sensor is further adapted to provide, in real time, positioning information about the working end to the computer; and
the computer is further adapted to cause the control unit to control, in real time, the operation of the surgical tool in view of relative positions of the working end and of a given landmark indicative of the anatomical structure of interest.

25. The system of claim 23 or 24, wherein the computer is further adapted to:
compare, in real time, the path of the surgical tool with a path defined in a treatment plan corresponding to the surgical procedure; and
evaluate, in real time, a compliance of the path of the surgical tool with the path defined in the treatment plan.

26. The system of claim 25, wherein the computer is further adapted to cause the control unit to stop operation of the surgical tool when the path of the surgical tool does not comply with the path defined in the treatment plan.

27. The system of claim 25, wherein the computer is further adapted to cause the control unit modify a trajectory of the surgical tool when the path of the surgical tool does not comply with the path defined in the treatment plan.

28. The system of claim 25, wherein the computer is further adapted to cause the control unit modify an operating speed of the surgical tool when the path of the surgical tool does not comply with the path defined in the treatment plan.

29. The system of any one of claims 25 to 28, wherein the computer is further adapted to cause the display device to display a warning sign when the path of the surgical tool does not comply with the path defined in the treatment plan.

30. The system of any one of claims 25 to 29, wherein the treatment plan includes a dental prosthetic plan and the path is defined in the treatment plan in view of improving at least one of a function and an appearance of a dental restoration.

31. The system of claim 30, wherein the treatment plan is based at least in part on an intraoral surface scan.

32. The system of claim 30 or 31, wherein:
the surgical tool comprises a drill;
the anatomical structure of interest includes a mandible or a maxilla of a patient; and
the dental prosthetic plan includes inserting an end of an implant in the mandible or the maxilla of the patient and mounting a prosthesis on an opposite end of the implant.

33. The system of any one of claims 1 to 32, wherein the display device is a head-mountable display.

34. The system of claim 33, wherein:
the head-mountable display comprises a field-of-view (FOV) camera operatively connected to the computer and adapted to provide images of the field of view of the user to the computer; and
the computer is adapted to create the augmented reality view further based on the images of the field of view of the user.

35. The system of claim 34, wherein the computer is further adapted to cause the display device to display the augmented reality view when the computer detects that the anatomical structure of interest is within the field of view of the user.

36. The system of claim 34 or 35, wherein the computer is further adapted to cause the display device to display a virtual reality view of the anatomical structure of interest when the computer detects that the anatomical structure of interest is not within the field of view of the user.

37. The system of claim 34, wherein the computer is further adapted to cause the display device to display a virtual reality view of a predicted outcome of the surgical procedure when the computer detects that the anatomical structure of interest is not within the field of view of the user.

38. The system of any one of claims 1 to 36, wherein the computer is further adapted to:
predict, in real time, an outcome of the surgical procedure; and
include a view of the predicted outcome of the surgical procedure in the augmented reality view.

39. A method for assisting a user in a surgical procedure, comprising:
acquiring three dimensional (3D) positioning information about an anatomical structure of interest;
acquiring 3D positioning information about a surgical tool;
determining a path of the surgical tool;
creating, based on the 3D positioning information, an augmented reality view of spatial relations between the anatomical structure of interest and one or more positions along the path of the surgical tool; and
displaying the augmented reality view superimposed over a field of view of the user.

40. The method of claim 39, wherein the surgical procedure is planned in view of improving at least one of a function and an appearance of a dental restoration.

41. The method of claim 39 or 40, wherein the path of the surgical tool is determined at least in part based on a current position of the surgical tool.

42. The method of any one of claims 39 to 41, wherein the path of the surgical tool is further determined at least in part based on a previous position of the surgical tool.

43. The method of any one of claims 39 to 42, wherein the path of the surgical tool is further determined at least in part based on a predicted position of the surgical tool calculated based on a current position of the surgical tool and on a current orientation of the surgical tool.

44. The method of any one of claims 39 to 43, wherein acquiring the 3D positioning information about the anatomical structure of interest comprises acquiring 3D positioning information about a plurality of landmarks indicative of the anatomical structure of interest.

45. The method of any one of claims 39 to 44, further comprising:
positioning at least one fiducial marker proximally to the anatomical structure of interest; and
acquiring 3D positioning information about the at least one fiducial marker;
wherein the augmented reality view is created further based on the 3D positioning information about the at least one fiducial marker.

46. The method of claim 45, wherein the at least one fiducial marker is an infrared emitter, acquiring 3D positioning information about the at least one fiducial marker comprising using an infrared detector.

47. The method of claim 45 or 46, wherein the at least one fiducial marker comprises a plurality of fiducial markers, the method further comprising triangulating the 3D positioning information about the plurality of markers.

48. The method of any one of claims 45 to 47, wherein:
one of the at least one fiducial marker is a 3D fiducial structure; and
acquiring 3D positioning information about the at least one fiducial marker comprises acquiring 3D positioning information about a plurality of reference points on the 3D fiducial structure.

49. The method of any one of claims 45 to 48, further comprising:
acquiring a 3D map of the anatomical structure of interest and of the at least one fiducial marker;
wherein the augmented reality view is created further based on the 3D map.

50. The method of claim 49, wherein:
the 3D map is acquired, before the surgical procedure, from an apparatus selected from a computerized tomography (CT) scanner, a cone beam computed tomography (CBCT) scanner and a magnetic resonance imaging (MRI) device; and
the 3D map is stored in a database for access thereto during the surgical procedure.

51. The method of claim 50, further comprising:
using a 3D camera to capture images of the anatomical structure of interest, of the at least one fiducial marker, and of the surgical tool; and
performing a registration between images captured by the 3D camera and a content of the 3D map to update the 3D map.

52. The method of claim 49, further comprising:
using a 3D camera to capture images of the anatomical structure of interest, of the at least one fiducial marker and of the surgical tool; and
generating the 3D map based on the captured images.

53. The method of any one of claims 45 to 52, further comprising:
acquiring a 3D map of the anatomical structure of interest;
wherein the augmented reality view is created further based on the 3D map.

54. The method of claim 53, wherein:
the 3D map is acquired, before the surgical procedure, from an apparatus selected from a computerized tomography (CT) scanner, a cone beam computed tomography (CBCT) scanner and a magnetic resonance imaging (MRI) device; and
the 3D map is stored in a database for access thereto during the surgical procedure.

55. The method of claim 54, further comprising:
using a 3D camera to capture images of the anatomical structure of interest and of the surgical tool; and
performing a registration between images captured by the 3D camera and a content of the 3D map to update the 3D map.

56. The method of claim 53, further comprising: using a 3D camera to capture images of the anatomical structure of interest and of the surgical tool; and
generating the 3D map based on the captured images.

57. The method of any one of claims 49 to 56, wherein the 3D map comprises a plurality of voxels distributed over three dimensions, each voxel having at least one intensity value and a coordinate over each of the three dimensions.

58. The method of claim 57, wherein each voxel has at least one polychromatic value, the at least one polychromatic value being derivable from the at least one intensity value.

59. The method of any one of claims 49 to 58, wherein the 3D map comprises position, orientation and scale information of features of the anatomical structure of interest.

60. The method of any one of claims 49 to 59, further comprising:
selecting elements of the 3D map representing at least one cross-section of the anatomical structure of interest;
wherein the augmented reality view is created further based on the at least one cross-section of the anatomical structure of interest.

61. The method of any one of claims 39 to 60, wherein the 3D positioning information about the about the anatomical structure of interest and the 3D positioning information about the surgical tool are acquired in real time, the method further comprising:
determining, in real time, spatial relations between the one or more positions along the path of the surgical tool and a position of a given landmark indicative of the anatomical structure of interest; and
controlling, in real time, an operation of the surgical tool in view of the spatial relations between the one or more positions along the path of the surgical tool and the position of the given landmark indicative of the anatomical structure of interest.

62. The method of claim 61, wherein the surgical tool comprises a working end, the method further comprising:
acquiring, in real time, positioning information about the working end;
determining, in real time, relative positions of the working end of the surgical tool and of a given landmark indicative of the anatomical structure of interest; and
controlling, in real time, an operation of the surgical tool in view of the relative positions of the working end of the surgical tool and of the given landmark indicative of the anatomical structure of interest.

63. The method of claim 61 or 62, further comprising:
tracking, in real time, a progression of the position of the surgical tool;
comparing, in real time, the path of the surgical tool with a path defined in a treatment plan corresponding to the surgical procedure; and
evaluating, in real time, a compliance of the path of the surgical tool with the path defined in the treatment plan.

64. The method of claim 63, further comprising stopping operation of the surgical tool when the path of the surgical tool does not comply with the path defined in the treatment plan.

65. The method of claim 63, further comprising modifying a trajectory of the surgical tool when the path of the surgical tool does not comply with the path defined in the treatment plan.

66. The method of claim 63, further comprising modifying an operating speed of the surgical tool when the path of the surgical tool does not comply with the path defined in the treatment plan.

67. The method of any one of claims 63 to 66, further comprising displaying a warning sign when the path of the surgical tool does not comply with the path defined in the treatment plan.

68. The method of any one of claims 63 to 67, wherein the treatment plan includes a dental prosthetic plan.

69. The method of claim 68, wherein the treatment plan is based at least in part on an intraoral surface scan.

70. The method of claim 68 or 69, wherein:
the surgical tool comprises a drill;
the anatomical structure of interest includes a mandible or a maxilla of a patient; and
the dental prosthetic plan includes inserting an end of an implant in the mandible or the maxilla of the patient and mounting a prosthesis on an opposite end of the implant.

71. The method of any one of claims 39 to 70, further comprising using a head-mountable display to display the augmented reality view superimposed over the field of view of the user.

72. The method of claim 71, further comprising:
using a field-of-view (FOV) camera to acquire images of the field of view of the user;
wherein the augmented reality view is created further based on the images of the field of view of the user.

73. The method of any one of claims 39 to 72, further comprising displaying a predicted outcome of the surgical procedure as a part of the augmented reality view superimposed over the field of view of the user.

74. The method of claim 73, wherein the predicted outcome is calculated based on the path of the surgical tool.

75. Use of the system of any one of claims 1 to 38 to assist an implantology procedure in dental surgery.

76. Use of the method of any one of claims 39 to 74 to assist an implantology procedure in dental surgery.
